(19) United States
(12) Patent Application Publication
(10) Pub. No.: US 2025/0268572 A1
(43) Pub. Date: Aug. 28, 2025

(54) ANIMAL ULTRASOUND TRANSDUCER

(71) Applicant: OrcaWest Holdings, LLC, Loveland, CO (US)

(72) Inventors: Nathanael Alexander, Loveland, CO (US); Peter Temple, Loveland, CO (US); Matthew Todd Spigelmyer, Loveland, CO (US)

(21) Appl. No.: 18/590,322

(22) Filed: Feb. 28, 2024

(51) Int. Cl. A61B 8/00

(52) U.S. Cl. CPC A61B 8/4427; A61B 8/4455; A61B 8/4494; A61B 2503/40

(57) ABSTRACT

An ultrasound transducer for animals employs a crystal array with a novel combination of length, curvature, width, focus, operating frequency, and crystal pitch to optimize sonogram detail, resolution, field of view, and depth of view. The transducer includes a probe housing having a convex animal-contacting wall; and an array of piezoelectric crystals positioned along the convex wall. Properties of the transducer probe provide a shorter transducer length and smaller crystal element pitch to provide superior near field resolution, a more gradual resolution loss, and superior patient contact.

### BACKGROUND

An ultrasound transducer, also called a probe, is a device that produces sound waves that bounce off body tissues and make echoes. The transducer receives the echoes and sends them to a computer that uses them to create an image called a sonogram. Such transducers are frequently used by veterinarians and others in the animal industry to inspect large animal reproductive systems and other body parts.

The two most common types of transducers for large animal reproductive systems are flat linear transducers and curved transducers. The most important differences between them are their fields of view and frequency ranges.

Flat linear transducers typically operate in the 5-9 MHz range and produce a square or rectangular-shaped image with a penetration of about 15 cm below the surface, whereas curved transducers typically operate in the 3-7 MHz range and produce a larger wedge-shaped image with a penetration of about 24 cm.

Both transducer types have advantages and disadvantages. Because the flat linear probe has a smaller field of view, structures within that imaging plane will appear larger on the screen. Its image stays more consistent and with less distortion as it reaches the far field, where the sound waves of a curved probe fan further apart and lateral resolution is impaired. In addition, the higher frequency range of the flat probe produces a higher resolution image. Therefore, the flat linear probe is considered a better choice for examining smaller, shallower structures like earlier pregnancies and ovaries with the finest detail possible. It's also the best choice for scanning foal and calf lungs, as the pleural surface is quite shallow, and the flat transducer makes the best contact in the intercostal spaces.

Ultimately, the flat linear probe is a better choice for image quality over depth of penetration. It is the superior option for examining ovaries, early pregnancies, and producing images for fetal gender determination in the classic 60-80 day window. Typical scenarios for a flat linear probe include dairy and equine reproduction, foal and calf lung screening, and transrectal applications in smaller species such as deer, camelids, and small ruminants, where the physical size of the transducer head are a consideration.

The curved probe was born from the need to examine later stages of animal pregnancy, when the flat linear probe simply can't image deep enough. The wide field of view and low frequency provides a much broader view of the reproductive tract, visualizing larger fetuses. In addition, the curved face inherently makes better contact with the rectal mucosa. Very little pressure or leverage is necessary to eliminate air and produce a consistent image.

The curved probe is ideal for beef operations, where the scanner might want to see as much of a later term pregnancy as possible. It allows for fetal aging and sexing later than the linear probe will. It's also a great choice for a mixed large animal veterinarian who regularly conducts pregnancy exams on both cattle and small ruminants, camelids, or swine and only wants to invest in a single probe for both transrectal and transabdominal use.

For mixed practices seeing a larger variety of animals, both transducers are often needed because both have limitations. But investing in and maintaining two transducers can be cost prohibitive, and deciding which to use can be difficult and time consuming.

### SUMMARY

The present invention solves the above-described problems and provides a distinct advance in the art of ultrasound transducers for animals by providing a transducer that has many of the benefits of both flat and curved probes with few of the limitations of either.

The transducer of the present invention is not just a hybrid or “in-betweener”, but instead employs novel properties that together achieve the details and resolution of a flat probe and the larger field of view and depth of a curved probe. An embodiment of the transducer employs a crystal array with a novel combination of length, curvature, width, focus, operating frequency, and crystal pitch to optimize sonogram detail, resolution, field of view, and depth of view.

The transducer of the present invention broadly comprises a probe housing having a convex animal-contacting wall and an array of piezoelectric crystals positioned along the convex wall. An embodiment of the crystal array has an active length of less than 60 mm, a radius of curvature of at least 62 mm, an active elevation width of between 7-9 mm, a unique sensor pitch of 400 um, and an operating frequency range of 3 MHz-9.5 MHz. These properties together, which are described in detail below along with other properties of the transducer, provide superior near field resolution, a more gradual resolution loss, and superior patient contact. Because the transducer of the present invention has many of the benefits of both flat and curved probes with few of the limitations of either, it can replace both probes so that a user only has to invest in and maintain one transducer probe.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a top front perspective view of an ultrasound transducer for animals constructed in accordance with embodiments of the present invention.

FIG. 2 is a bottom front perspective view of the ultrasound transducer with the crystal array removed from the housing.

FIG. 3 is a top rear perspective view of the ultrasound transducer.

FIG. 4 is a bottom rear perspective view of the ultrasound transducer with the crystal array removed from the housing.

FIG. 5 is a front elevational view of the transducer.

FIG. 6 is a side elevational view of the transducer.

FIG. 7 is a front elevational view of a prior art transducer.

FIG. 8 is a side elevational view of the prior art transducer of FIG. 7

FIG. 9 is a front elevational view of another prior art transducer.

FIG. 10 is a side elevational view of the prior art transducer of FIG. 9.

FIG. 11 is a side elevation view of the transducer of the present invention with the crystal array attached to the housing.

FIGS. 12 is a bottom perspective view of the transducer of the present invention with the crystal array attached to the housing.

FIGS. 13 is another bottom perspective view of the transducer of the present invention with the crystal array attached to the housing.

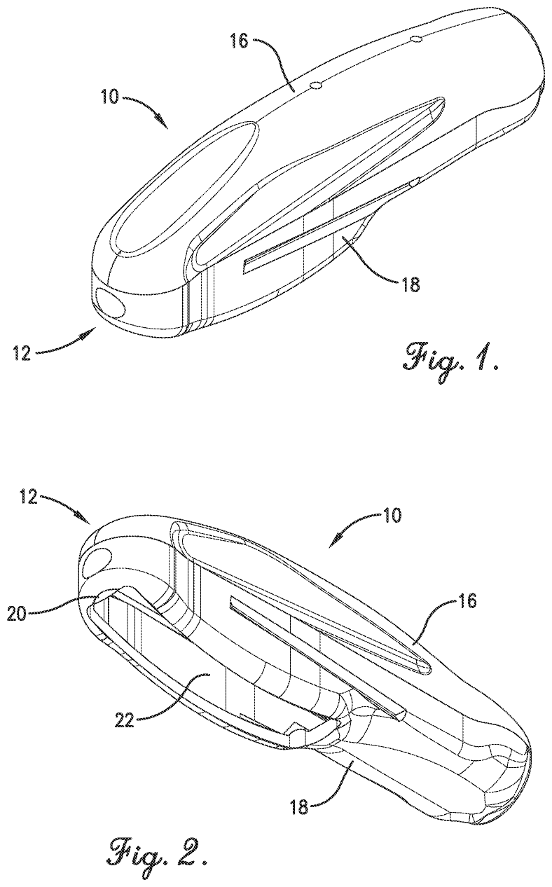

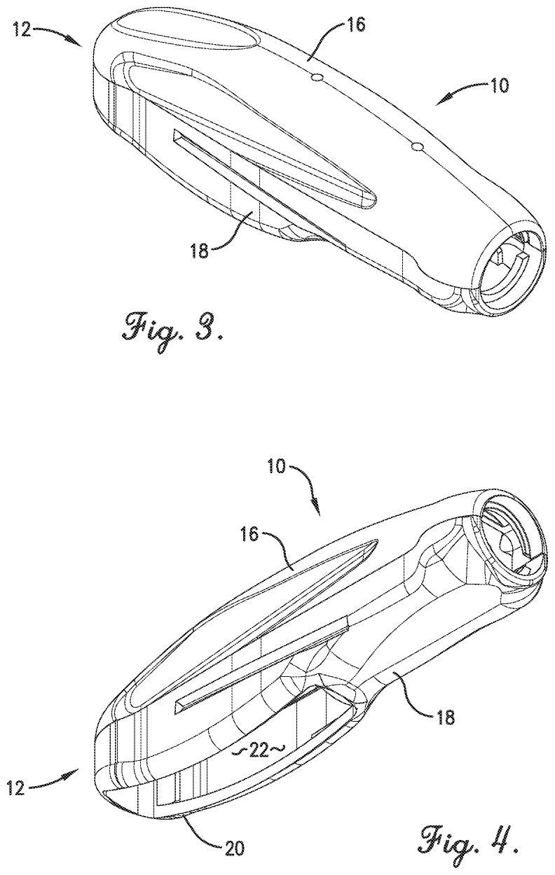

The drawing figures do not limit the present invention to the specific embodiments disclosed and described herein. The drawings are not necessarily to scale, emphasis instead being placed upon clearly illustrating the principles of the invention.

### DETAILED DESCRIPTION

An ultrasound transducer 10 constructed in accordance with embodiments of the present invention is illustrated in the attached drawing figures. As described in more detail below, embodiments of the transducer 10 have a novel combination of length, curvature, width, focus, operating frequency, and crystal pitch to optimize sonogram detail, resolution, field of view, depth of view, and patient contact.

Embodiments of the transducer 10 broadly comprise a probe housing 12 and an array 14 of piezoelectric crystals positioned within the housing. The transducer also includes or is coupled to a cable and connector for connecting to other sonogram equipment, which are not described in detail in this application. The transducer may also be attached to an elongated handle for facilitating its insertion in a body cavity of an animal.

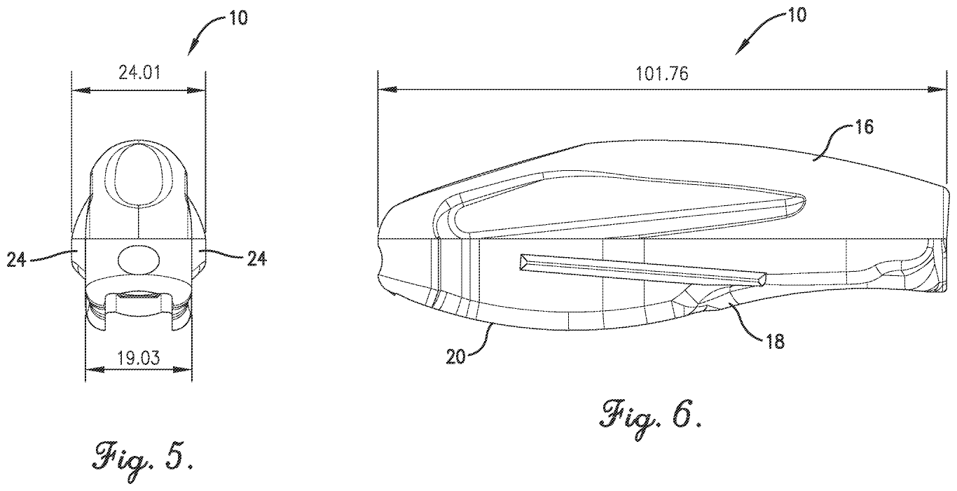

The housing 12 may be formed of plastic or other suitable materials, and in one embodiment, has upper and lower connecting sections 16, 18. As best shown in FIG. 2, the lower section 18 has a convex animal-contacting wall 20 with an opening 22 for receiving and supporting the crystal array 14. The upper section 16 is ergonomically curved and has a pair of enlarged grips 24 (See FIG. 5) for facilitating easy handling by a user.

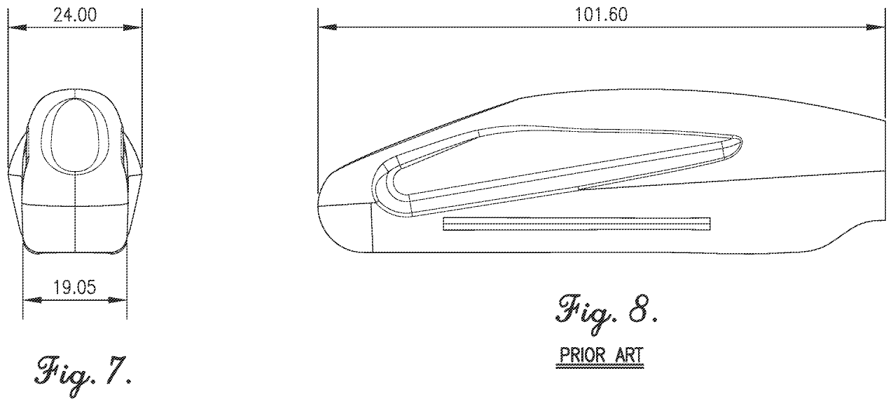

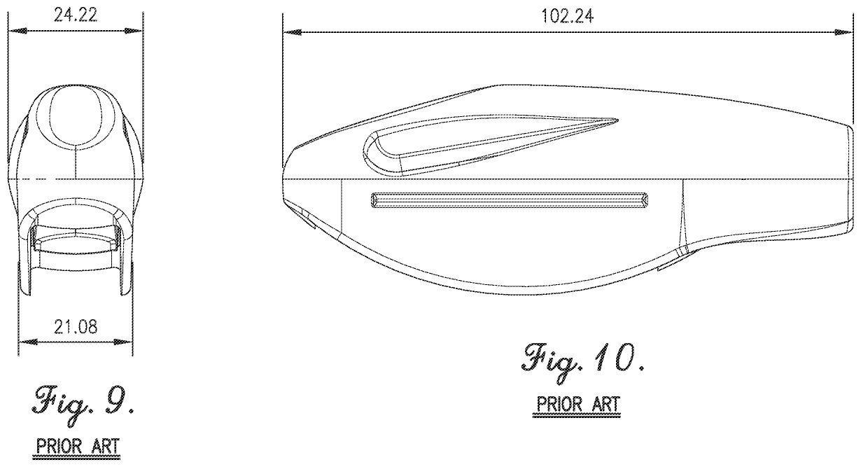

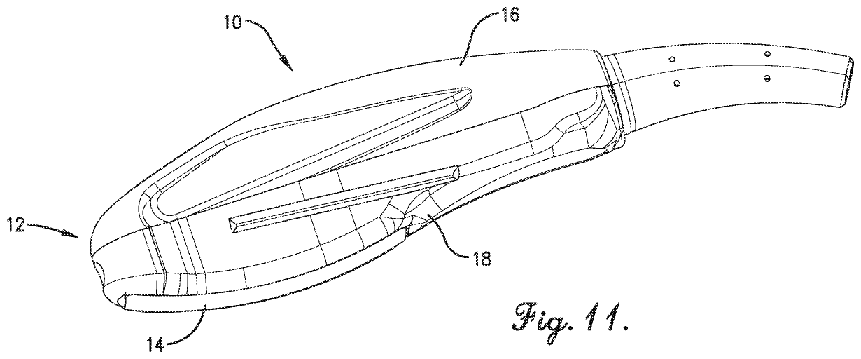

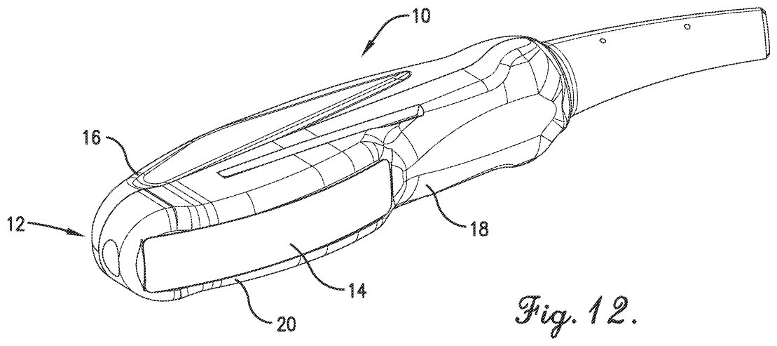

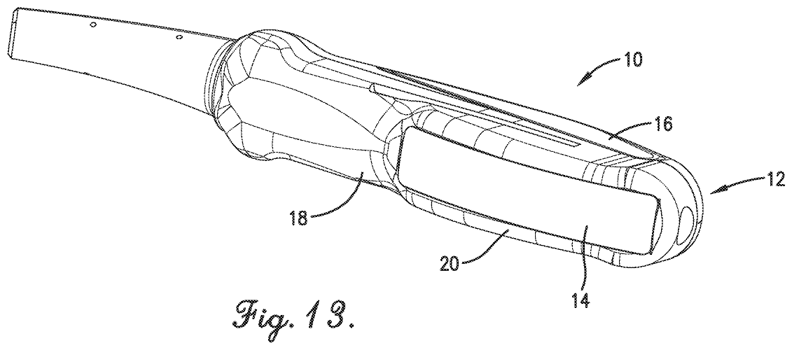

As shown in FIGS. 5 and 6, an embodiment of the housing is 24.01 mm wide including the grips 24, 19.03 mm wide not including the grips, and 101.7 mm long. For comparison, a prior art flat transducer is shown in FIGS. 7 and 8 and a prior art curved transducer is shown in FIGS. 9 and 10. The prior art flat transducer is 24 mm wide including the grips, 19.05 mm wide not including the grips, and 101.6 mm long. The prior art curved transducer is 24.22 mm wide including the grips, 21.08 mm wide not including the grips, and 102.4 mm long. The novel aspects of the crystal array described herein allow the transducer housing 12 to be approximately the same size as the prior art flat transducer shown in FIGS. 7 and 8 and much smaller than the prior art curved transducer shown in FIGS. 9 and 10.

The crystal array 14 will now be described in more detail. The array 14 is positioned in the curved animal contacting wall 20 of the housing and includes a number of aligned crystal elements. In one embodiment, the crystal array has 128 crystal elements, but it may have more or less crystal elements without departing from the scope of the invention. In one embodiment, the crystal elements are arranged in a row or linearly, but they may also be arranged in other ways.

The crystal array 14 has a number of properties that in combinations and sub combinations optimize the transducer performance to achieve the details and resolution of a flat probe and the larger field of view and depth of a curved probe to provide superior sonogram detail, resolution, field of view, and depth of view. An embodiment of the crystal array specifically provides a shorter transducer length and smaller crystal element pitch to provide superior near field resolution, a more gradual resolution loss, and superior patient contact.

An embodiment of the array 14 has an active length of less than 60 mm, where the active length is measured as the combined length of all the crystal elements. In one embodiment, the crystal array 14 has an active length of 51.2 mm. In comparison, the active length of the crystal array in the prior art flat transducer of FIGS. 7 and 8 is nearly 67 mm, and the active length of the crystal array in the prior art curved transducer of FIGS. 9 and 10 is nearly 61 mm.

An embodiment of the array 14 has an overall length of less than 60 mm, where the overall length is measured as the total length of all the crystal elements and the unused ends of the array. In one embodiment, the crystal array has an overall length of 52.88 mm. In comparison, the overall length of the crystal array in the prior art flat transducer of FIGS. 7 and 8 is nearly 69 mm, and the overall length of the crystal array in the prior art curved transducer of FIGS. 9 and 10 is nearly 62 mm.

An embodiment of the array has a radius of curvature of at least 62 mm. In one embodiment, the crystal array has a radius of curvature of 80.2 mm. In comparison, the prior art flat transducer of FIGS. 7 and 8 has no curvature, and the radius of curvature of the crystal array in the prior art curved transducer of FIGS. 9 and 10 is nearly 61 mm.

An embodiment of the array has an active elevation width of between 7-9 mm, where the active elevation width is measured as the width of the active portions of the crystal array. In one embodiment, the crystal array has an active elevation width of 8.5 mm. In comparison, the active elevation width of the crystal array in the prior art flat transducer of FIGS. 7 and 8 is nearly 6.5 mm, and the active elevation width of the crystal array in the prior art curved transducer of FIGS. 9 and 10 is 10 mm.

An embodiment of the array has a sensor pitch of less than 470 um, where the pitch is measured as the width of each crystal element and therefore the distance between the center points of adjacent crystal elements. In one embodiment, the crystal array has a pitch of 400 um. In comparison, the pitch of the crystal array in the prior art flat transducer of FIGS. 7 and 8 is 520 um, and the pitch of the crystal array in the prior art curved transducer of FIGS. 9 and 10 is 475 um.

An embodiment of the array has a focus depth of 60 mm. In comparison, the crystal array in the prior art flat transducer of FIGS. 7 and 8 has a focus depth of 40 and the crystal array in the prior art curved transducer of FIGS. 9 and 10 has a focus depth of 60 mm.

An embodiment of the array has an operating frequency range of 3 MHz-9.5 MHz. In comparison, the crystal array in the prior art flat transducer of FIGS. 7 and 8 has an operating frequency range of 3.85 MHz-10.25 MHz and the crystal array in the prior art curved transducer of FIGS. 9 and 10 has an operating frequency range of 2.5 MHz-7.75 MHz.

The above-described properties, alone and in combinations and sub-combinations, optimize the performance of the transducer 10. For example, the array's shorter length and pitch provide better near field resolution, and the array's radius of curvature provides a more gradual loss of resolution and better patient contact versus prior art transducers. Because the transducer of the present invention has many of the benefits of both flat and curved probes with few of the limitations of either, it can replace both probes so that a user only has to invest in and maintain one transducer probe.

### ADDITIONAL CONSIDERATIONS

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. An ultrasound transducer for animals, the transducer comprising:
a probe housing having a convex animal-contacting wall; and
an array of piezoelectric crystals positioned along the convex animal-contacting wall of the probe housing, the array having an active length of less than 60 mm, a radius of curvature of at least 62 mm, and an active elevation width of between 7-9 mm.

2. The ultrasound transducer of claim 1, wherein the array has an active length of 51.2 mm.

3. The ultrasound transducer of claim 1, wherein the array has a radius of curvature of 80.2 mm.

4. The ultrasound transducer of claim 1, wherein the array has an active elevation width of 8.5 mm.

5. The ultrasound transducer of claim 1, wherein the array has a focus depth of 60 mm.

6. The ultrasound transducer of claim 1, wherein the array has a pitch of less than 475 um.

7. The ultrasound transducer of claim 1, wherein the array has a pitch of 400 um.

8. The ultrasound transducer of claim 1, wherein the array has a frequency of 3 MHz-9.5 MHz.

9. An ultrasound transducer for animals, the transducer comprising:
a probe housing having a convex animal-contacting wall; and
an array of piezoelectric crystals positioned along the convex animal-contacting wall of the probe housing, the array having a pitch of less than 475 um.

10. The ultrasound transducer of claim 9, wherein the array has an active length of less than 60 mm, a radius of curvature of at least 62 mm, and an active elevation width of between 7-9 mm.

11. The ultrasound transducer of claim 9, wherein the array has an active length of 51.2 mm.

12. The ultrasound transducer of claim 9, wherein the array has a radius of curvature of 80.2 mm.

13. The ultrasound transducer of claim 9, wherein the array has an active elevation width of 8.5 mm.

14. The ultrasound transducer of claim 9, wherein the array has a focus depth of 60 mm.

15. The ultrasound transducer of claim 9, wherein the array has a pitch of less than 475 um.

16. The ultrasound transducer of claim 9, wherein the array has a pitch of 400 um.

17. The ultrasound transducer of claim 9, wherein the array has a frequency of 3 MHz-9.5 MHz.

18. An ultrasound transducer for animals, the transducer comprising:
a probe housing having a convex animal-contacting wall;
an array of piezoelectric crystals positioned along the convex animal-contacting wall of the probe housing, the array having an active length of less than 60 mm, a radius of curvature of at least 62 mm, and an active elevation width of between 7-9 mm, and a pitch of less than 475 um.

19. The ultrasound transducer of claim 18, wherein the array has an active length of 51.2 mm, a radius of curvature of 80.2 mm, an active elevation width of 8.5 mm, a focus depth of 60 mm and a pitch of 400 um.

20. The ultrasound transducer of claim 18, wherein the array has a frequency of 3 MHz-9.5 MHz.
